(19) United States
(12) Patent Application Publication
(10) Pub. No.: US 2024/0390533 A1
(43) Pub. Date: Nov. 28, 2024

(54) DISINFECTING LUMINAIRE USING SWITCHABLE MIRRORS

(71) Applicant: SIGNIFY HOLDING B.V., EINDHOVEN (NL)

(72) Inventors: VOYA VIDAKOVIC, MARIETTA, GA (US); NAM CHIN CHO, PEACHTREE CITY, GA (US)

(21) Appl. No.: 18/294,218

(22) PCT Filed: Jul. 20, 2022
(86) PCT No.: PCT/EP2022/070345

§ 371 Date: Feb. 1, 2024

(30) Foreign Application Priority Data
Aug. 16, 2021 (EP) 21191498.1

(51) Int. Cl. A61L 2/10; A61L 2/26; A61L 9/20; G01J 5/00

(52) U.S. Cl. CPC A61L 2/10; A61L 2/26; A61L 9/20; G01J 5/0025; A61L 2202/11; A61L 2202/121; A61L 2202/123; A61L 2202/14; A61L 2202/25; A61L 2209/111; A61L 2209/12; A61L 2209/15

(57) ABSTRACT

A disinfecting light apparatus configurable through the use of switchable mirror panels, occupancy sensors, and reflective louvers is disclosed. The disinfecting light apparatus controls the emission of disinfecting light using switchable mirror panels. The occupancy sensors generate occupancy signals used to determine the dimming level of the switchable mirror panels. The reflective louvers are configured to direct disinfecting light 5horizontally to disinfect the “upper-air.” The switchable mirror panels, occupancy sensors, and reflective louvers allow for the disinfecting light apparatus to switch between different modes. For example, if the occupancy sensors do not detect any persons in their fields of view, the disinfecting light apparatus operates in a “Surface Disinfection” mode. If the occupancy sensors detect a person, the disinfecting light apparatus operates in “Upper-Air 10Disinfection” mode. Alternatively, the disinfecting light apparatus can be configured to selectively dim individual switchable mirror panels depending on the location of detected persons.

### FIELD OF THE DISCLOSURE

The present disclosure is directed generally to a disinfecting luminaire using switchable mirrors.

### BACKGROUND

Disinfecting luminaires are becoming more common due to the recent COVID-19 pandemic. These disinfecting luminaires often use non-dimmable, non-directional fluorescent light sources. Safety guidelines limit the level of certain types of disinfecting light in certain situations, particularly when persons are present in the vicinity of the disinfecting light. As such, it is often necessary to power off the disinfecting luminaire in these situations. Continuously turning a disinfecting fluorescent light on and off in response to the detection of one or more persons may degrade the light and shorten its lifespan. Accordingly, there is a need in the art for controlling the intensity and direction of a disinfecting fluorescent light without powering the light on and off.

### SUMMARY OF THE DISCLOSURE

The present disclosure is generally directed to a disinfecting light apparatus configurable through the use of switchable mirror panels and occupancy sensors. The switchable mirror panels enable the disinfecting light apparatus to operate in a variety of different modes depending on the data collected by the occupancy sensors.

The disinfecting light apparatus includes a disinfecting light source generating disinfecting light, such as a non-dimmable fluorescent lamp. The disinfecting light apparatus controls the emission of the disinfecting light using switchable mirror panels. The switchable mirror panels can fluctuate between 0 and 100 percent dimming based on voltage applied to its leads. The disinfecting light apparatus further includes one or more occupancy sensors. The occupancy sensors generate occupancy signals used to determine the dimming level of the switchable mirror panels. The disinfecting light apparatus further includes one or more reflective louvers. The reflective louvers are configured to direct disinfecting light horizontally to disinfect the “upper-air” around the disinfecting light apparatus.

The switchable mirror panels, occupancy sensors, and reflective louvers allow for the disinfecting light apparatus to switch between a number of different modes. For example, if the occupancy sensors do not detect any persons in their respective fields of view, the disinfecting light apparatus may operate in a “Surface Disinfection” mode. If the occupancy sensors detect a person, the disinfecting light apparatus may operate in “Upper-Air Disinfection” mode. As an alternative to Upper-Air Disinfection mode, the disinfecting light apparatus can be configured to selectively dim individual switchable mirror panels depending on the location of detected persons.

Generally, in one aspect, a disinfecting light apparatus is provided. According to an example, the disinfecting light apparatus is approximately octagonal.

The disinfecting light apparatus includes a disinfecting light source. The disinfecting light source is configured to generate disinfecting light. According to an example, the disinfecting light is GUV light.

The disinfecting light apparatus further includes one or more occupancy sensors. Each occupancy sensor is configured to generate an occupancy signal. At least one of the one or more occupancy sensors are passive infrared (PIR) sensors.

The disinfecting light apparatus further includes one or more reflective louvers. The one or more reflective louvers are configured to direct the disinfecting light horizontally.

The disinfecting light apparatus further includes one or more switchable mirror panels. Each switchable mirror panel is configured to dim according to at least one of the one or more occupancy signals. According to an example, the disinfecting light apparatus includes 5 to 7 switchable mirror panels.

According to an example, the disinfecting light apparatus further includes one or more pendant mounts. The pendant mounts are configured to mount the disinfecting light apparatus to a ceiling.

According to an example, the one or more reflective louvers are positioned adjacent to at least one of the one or more switchable mirror panels. The one or more reflective louvers are arranged at an angle of 89 to 91 degrees relative to the adjacent switchable mirror panel.

According to an example, the disinfecting light apparatus further includes a metal top cover. The metal top cover is adjacent to at least one of the one or more switchable mirror panels.

According to an example, the disinfecting light apparatus further includes a controller. The controller is configured to generate a panel control signal. The panel control signal is based on at least one of the one or more occupancy signals. The panel control signal sets the dimming level for at least one of the switchable mirror panels. According to a further example, the panel control signal sets the dimming level of the at least one of the one or more switchable mirror panels between 0 and 100 percent. According to an even further example, at least one of the one or more switchable mirror panels dim to 100 percent if at least one of the one or more occupancy signals indicates occupancy. The panel control signal may be further based on one or more safety guidelines and/or the intensity of the disinfecting light.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is an isometric view of a disinfecting light apparatus, in accordance with an example.

FIG. 2 is a cross-sectional view of a disinfecting light apparatus, in accordance with an example.

FIG. 3 is functional block diagram of a disinfecting light apparatus, in accordance with an example.

FIGS. 4A and 4B are illustrations of a lighting apparatus transitioning from Surface Disinfection mode to Upper-Air Disinfection mode, in accordance with an example.

FIGS. 5A and 5B are illustrations of a lighting apparatus transitioning from Surface Disinfection mode to Directional Disinfection mode, in accordance with an example.

FIG. 6 is a flowchart of a method for selectively dimming individual switchable mirror panels of a disinfecting light apparatus, in accordance with an example.

FIG. 7 is a flowchart of a method for transitioning a disinfecting light apparatus between Surface Disinfection mode and Upper-Air Disinfection mode, in accordance with an example.

### DETAILED DESCRIPTION OF EMBODIMENTS

The present disclosure is generally directed to a disinfecting light apparatus configurable through the use of switchable mirror panels and occupancy sensors. The switchable mirror panels enable the disinfecting light apparatus to operate in a variety of different modes depending on the data collected by the occupancy sensors.

The disinfecting light apparatus includes a disinfecting light source. The disinfecting light source is configured to generate disinfecting light which kills or inactivates viruses and/or bacteria. For example, the disinfecting light source may generate germicidal ultraviolet (GUV) light. GUV light, such as UV-C radiation, can be dangerous to humans. Accordingly, a number of safety guidelines exist for devices generating UV-C light to avoid injuring humans. In some examples, the disinfecting light source can be a non-dimmable fluorescent lamp. In further examples, the disinfecting light has a wavelength outside of the UV spectrum, such as 405 nanometers.

The disinfecting light apparatus controls the emission of disinfecting light using switchable mirror panels. The switchable mirror panels can fluctuate between 0 and 100 percent dimming based on voltage to its leads. At a dimming level of 0 percent, the switchable mirror panel acts as a transparent pain of glass, and approximately all incident disinfecting light will pass through the panel to the environment outside of the disinfecting light apparatus. At a dimming level of 100 percent, the switchable mirror panel acts as a mirror, and all incident disinfecting light is reflected back into the disinfecting light apparatus. Further, dimming levels between 0 and 100 percent (such as 50 percent) may be used to partially reflect the light incident to the switchable mirror panel, thus resulting in a dimming effect reducing the exposure of disinfecting light on nearby persons. Therefore, the switchable mirror panels can be used to control both the direction and quantity of disinfecting light emitted by the disinfecting light apparatus. The switchable mirror panels may be liquid crystal switchable mirrors.

Additionally, using switchable mirror panels allows for the control of the disinfecting light emissions without turning the disinfecting light source on and off. This is particularly advantageous if the disinfecting light source is a fluorescent lamp, as continually turning a fluorescent lamp on and off can degrade the lamp and shorten its lifespan.

In one configuration, the disinfecting light apparatus is approximately octagonal. In this configuration, the apparatus includes eight panels; a top metal panel and seven switchable mirror panels. As disinfecting light apparatuses are typically hung from a ceiling via one or more pendant mounts, the top metal panel may be required due to safety guidelines regarding disinfecting light incident upon the ceiling. In a further example of an octagonal disinfecting light apparatus, the upper three panels are metal panels, while the lower five panels are switchable mirror panels.

The disinfecting light apparatus further includes one or more occupancy sensors. The occupancy sensors generate occupancy signals used to determine the dimming level of the switchable mirror panels. The occupancy sensors are configured to monitor the areas and surfaces to be disinfected by the disinfecting light apparatus. In a preferred example, the occupancy sensors are passive infrared (PIR) sensors. In other examples, the occupancy sensors can utilize other types of technologies to detect occupancy or human presence, such as ultrasonic, optical camera, radar, etc.

The disinfecting light apparatus further includes one or more reflective louvers. The reflective louvers are configured to direct disinfecting light horizontally to disinfect the “upper-air” around the disinfecting light apparatus. The reflective louvers are typically made of aluminum. In the example of the octagonal disinfecting light apparatus, the reflective louvers are arranged adjacently to the side switchable mirror panels. In order to direct light horizontally, each reflective louver is arranged at an angle between 89 and 91 degrees relative to the adjacent side switchable mirror panel.

The switchable mirror panels, occupancy sensors, and reflective louvers allow for the disinfecting light apparatus to switch between a number of different modes. For example, if the occupancy sensors do not detect any persons in their respective fields of view, the disinfecting light apparatus operates in a “Surface Disinfection” mode. In Surface Disinfection mode, the lower three switchable mirror panels of an octagonal disinfecting light apparatus are set to a dimming level of 0 percent. Accordingly, in Surface Disinfection mode, all disinfecting light generated by the disinfecting light source is directed downwards to surfaces below the disinfecting light source.

If the occupancy sensors detect a person, the disinfecting light apparatus may operate in an “Upper-Air Disinfection” mode. In this mode, the lower three switchable mirror panels are set to a dimming level of 100 percent, while the side switchable mirror panels, adjacent to the reflective louvers, are set to a dimming level of 0 percent. Accordingly, in Upper-Air Disinfection mode, all disinfecting light is directed horizontally relative to the disinfecting light apparatus. Using the Upper-Air Disinfection mode prevents persons from being exposed to potentially harmful light without turning the disinfecting light source off.

As an alternative to Upper-Air Disinfection mode, the disinfecting light apparatus can be configured to selectively dim individual switchable mirror panels depending on the location of detected persons. For example, if a person is determined to be positioned to the right of the disinfecting light apparatus, the dimming levels of the switchable mirror panels on the right-side of the apparatus are set to 100 percent. In this way, the detected persons are protected from the disinfecting light, while other, non-occupied portions of the environment are disinfected. Further, intermediate dimming levels (such as 25, 50, or 75 percent) can be set to conform with safety guidelines regarding disinfecting light.

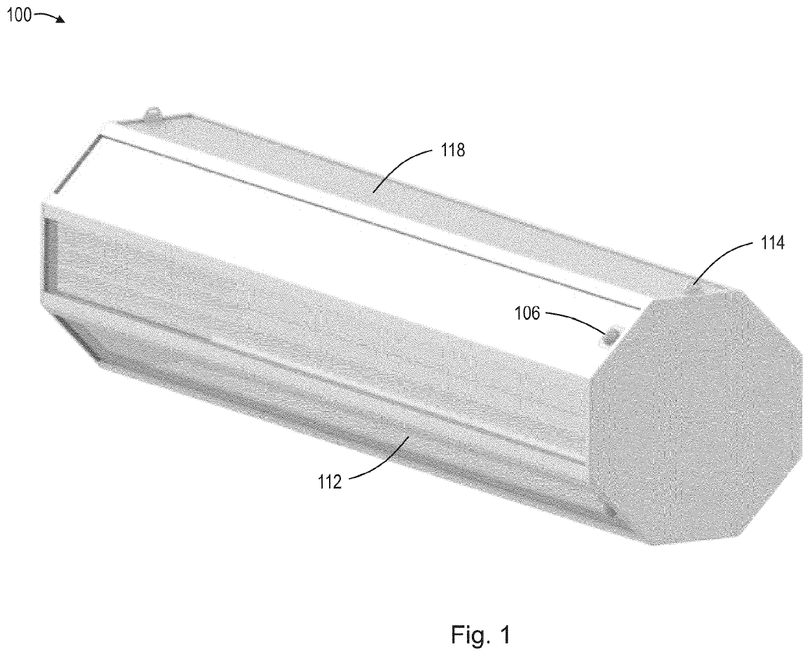

FIG. 1 shows an isometric view of an example disinfecting light apparatus 100. This disinfecting light apparatus 100 is embodied as a smart drum. The disinfecting light apparatus 100 of FIG. 1 is octagonal. However, it should be appreciated that the disinfecting light apparatus 100 can include other shapes, designs and/or dimensions based at least in part of a particular configuration of the disinfecting light apparatus 100 and/or an environment the disinfecting light apparatus 100 is installed or provided within. The upper three sides of the disinfecting light apparatus 100 are metal top covers 118. The metal top covers 118 are used to shield the ceiling 200 (upon which the disinfecting light apparatus 100 is mounted) from disinfecting light 104 generated by the disinfecting light source 102. This shielding may be used to meet industry and safety guidelines limiting the amount of disinfecting light 104 incident upon the ceiling 200. In some situations, the ceiling 200 can reflect incident disinfecting light 104 back into the environment to be disinfected in an unpredictable manner, making it difficult to evaluate and enforce safety guidelines 124 regarding disinfecting light 104.

The middle two longitudinal sides and lower longitudinal three sides of the disinfecting light apparatus 100 are switchable mirror panels 112. Each switchable mirror panel 112 is configured to dim based on an electrical signal applied to its leads. For example, the switchable mirror panel 112 could be configured to receive signals with voltages between 0 and 12 V and dim accordingly. At 0 V, the switchable mirror panel 112 operates at 0 percent dimming; at 6 V, the switchable mirror panel 112 operates at 50 percent dimming; and at 12 V, the switchable mirror panel 112 operates at 100 percent dimming. The switchable mirror panels 112 may also dim at levels between 0 and 6 V and/or 6 V and 12 V depending on the received signal. Further combinations of applied voltages and dimming levels are possible depending on the implementation.

At 0 percent dimming, the switchable mirror panel 112 is transparent, allowing incident light to pass through unimpeded. At 100 percent dimming, the switchable mirror panel 112 acts as a mirror, reflecting all incident light. At 50 percent dimming, half of the incident light is reflected, while the other half passes through the switchable mirror panel 112. At ranges between 0 percent dimming and 50 percent dimming, the switchable mirror panel 112 can reflect a percentage of incident light based at least in part on the respective percentage dimming level. At ranges between 50 percent dimming and 100 percent dimming, the switchable mirror panel 112 can reflect a percentage of incident light based at least in part on the respective percentage dimming level. The switchable mirror panel 112 can transition from 0 percent dimming to 100 percent dimming to provide a desired level of disinfectant or disinfecting light or block disinfecting light based at least in part on the presence of a person within an environment the disinfecting light apparatus 100 including the switchable mirror panel 112 is installed.

The dimming level 128 of each switchable mirror panel 112 is based on data collected by one or more occupancy sensors 106. FIG. 1 shows an occupancy sensor 106 affixed to the disinfecting light apparatus 100. As will be explained in greater detail below, the occupancy sensors 106 generate occupancy signals 108 representative of occupancy within their respective fields of view. The occupancy signals 108 are provided to a controller 120 for processing. The controller 120 then provides each switchable mirror panel 112 with a panel control signal 122. The panel control signal 122 may have a voltage between 0 V and 12 V used to set the dimming level 128 of the corresponding switchable mirror panel 112.

In a preferred embodiment, the occupancy sensors 106 are PIR sensors. In other examples, the occupancy sensors can utilize other types of technologies to detect occupancy or human presence, such as ultrasonic, optical camera, radar, etc. The occupancy sensors 106 can be positioned on the disinfecting light source 100 in any practical way to monitor occupancy of the surfaces to be disinfected.

The switchable mirror panels 112 and the top metal covers 118 surround disinfecting light source 102 (not shown). The disinfecting light source 100 is configured to generate disinfecting light 104 which kills or inactivates viruses and/or bacteria. The disinfecting light source may generate GUV light. GUV light, such as UV-C radiation, can be dangerous to humans. Accordingly, a number of safety guidelines 124 exist for devices generating UV-C light to avoid injuring humans. In further examples, the disinfecting light 104 has a wavelength outside of the UV spectrum, such as 405 nanometers.

In some examples, the disinfecting light source 102 can be a non-dimmable fluorescent lamp. Using switchable mirror panels allows 112 for the control of the disinfecting light 104 emissions without turning the disinfecting light source 102 on and off. As continually turning a fluorescent lamp on and off can degrade lamp and shorten its lifespan, allowing the fluorescent lamp to remain powered on while still controlling the emitted disinfecting light 104 is particularly advantageous.

FIG. 1 further shows two pendant mounts 114. In this example, these pendant mounts 114 are configured to affix or suspend the disinfecting light apparatus 100 to a ceiling 200 or other surface.

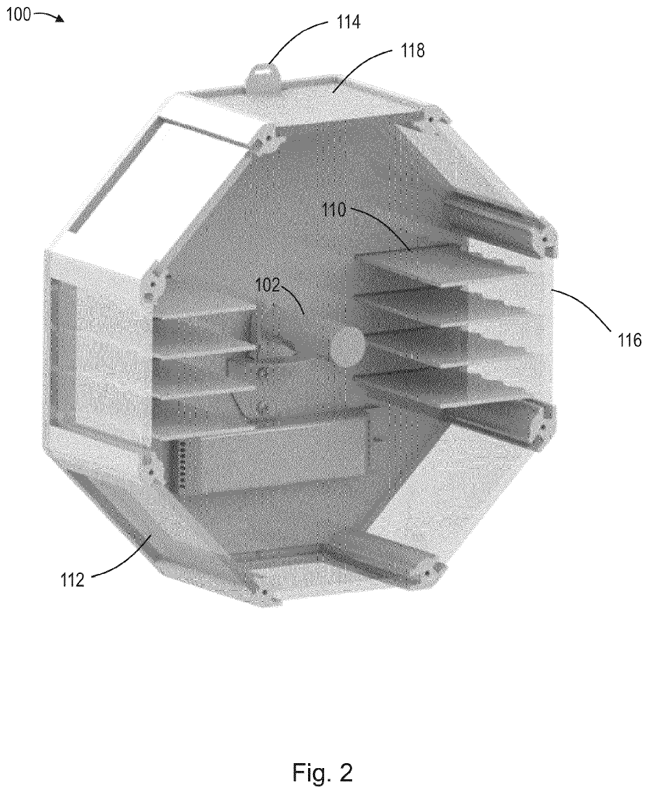

FIG. 2 shows a cross-sectional view of an example disinfecting light assembly 100. This view shows the interior of the disinfecting light apparatus 100 shown in FIG. 1. Notably, FIG. 2 shows a total of eight reflective louvers 110. It should be appreciated that the disinfecting light apparatus 100 can include fewer than eight reflective louvers 110 or more than eight reflective louvers 110 based at least in part on the shape, size and/or dimensions of the disinfecting light apparatus 100 and/or the number of switchable mirror panels 116 provided in the disinfecting light apparatus 100. Each of the reflective louvers 110 is positioned adjacent to a switchable mirror panel 116. The reflective louvers 110 are configured to direct disinfecting light 104 horizontally to disinfect the “upper-air” around the disinfecting light apparatus 102. The reflective louvers 110 are typically made of aluminum. In the example of the octagonal disinfecting light apparatus 100 shown in FIG. 2, the reflective louvers 110 are arranged adjacently to the side switchable mirror panels 116. In order to direct light horizontally, each reflective louver 110 is arranged at an angle between 89 and 91 degrees relative to the adjacent switchable mirror panel 116.

FIG. 2 also shows a cross-section of a disinfecting light source 102. The disinfecting light source 102 is mounted to the disinfecting light apparatus 100 via a bracket and socket configuration.

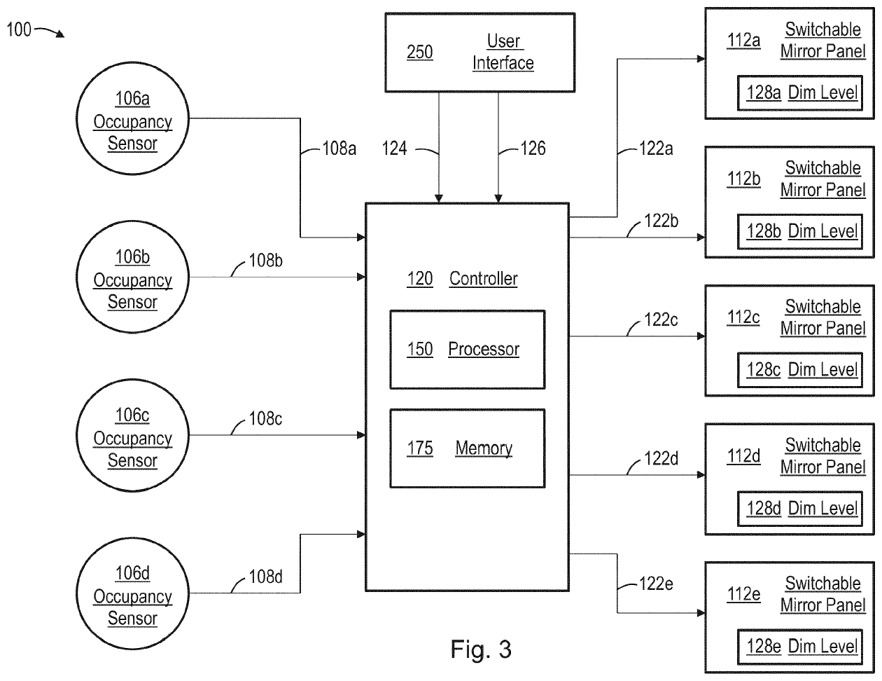

FIG. 3 shows a functional block diagram of a disinfecting light apparatus 100. FIG. 3 illustrates how data gathered from four occupancy sensors 106a-106d is used to control the dimming level 128a-128e of a group of five switchable mirror panels 112a-112e.

Each occupancy sensor 106 generates an occupancy signal 108. This occupancy signal 108 corresponds to an occupancy state within the field of view of the occupancy sensor 106. The occupancy signal 108 is provided to controller 120 for processing by processor 150. The occupancy signals 108 may also be stored in the memory 175 of the controller 120.

The controller 120 analyzes occupancy signals 108a-108d to determine if any persons are located within the fields of view of the occupancy sensors 106a-106d. Based on these determinations, the controller 120 can generate panel control signals 122a-122e for each of the switchable mirror panels 112a-112e. The controller 120 can generate panel control signals 122a-122e for a single switchable mirror panel 112 or two or more switchable mirror panels 112a-112e (e.g., groups of switchable mirror panels 112, a side portion of the disinfecting light apparatus 100 including two or more switchable mirror panels 112), for example, to target a specific area of a room or environment with disinfecting light or prevent disinfecting light from being applied to a specific area of a room or environment the disinfecting light apparatus 100 is installed. The panel control signals 122a-122e control the dimming level 128a-128e of each switchable mirror panel 112a-112e. The controller 120 can generate individual panel control signals 122 to control the dimming level 128 of individual switchable mirror panels 112 such that different switchable mirror panels 112 can receive different control signals 122 indicating different dimming levels 128. For example, the controller 120 can generate a first panel control signal 122 to control the dimming level 128 of a first individual switchable mirror panel 112 that is different from a second, different, panel control signal 122 used to control the dimming level 128 of a second, different switchable mirror panel 112. For example, if voltage of one panel control signal 122a is 0 V, the dimming level 128a is set to 0 percent, resulting in the corresponding switchable mirror panel 112a being transparent. The transparent switchable mirror panel 112a may be the result of the occupancy sensors 106a-106d to detect a person. In a further example, if the voltage of one panel control signal 122b is 12 V, the dimming level 128b is set to 100 percent, resulting in the corresponding switchable mirror panel 112b being a reflective mirror. The reflective switchable mirror panel 122b may be the result of one or more occupancy sensors 106a-106d detecting a person. Alternatively, the switchable mirror panel 122b may be reflective due to the controller 120 determining that the occupancy sensors 106a-106 detected a person within a field of view positioned to receive disinfecting light 104 shining through transparent 122b. The controller 120 can continually update or modify dimming levels 128 of one or more switchable mirror panels 112 using the panel control signals 122, for example, as one or more persons enter a field of view of an occupancy sensor 106, move around within a field of view of an occupancy sensor 106 and/or exit from a field of view of an occupancy sensor 106.

The controller 120 may process additional data in generating the panel control signals 122a-122e. For example, the controller 120 may receive information regarding a safety guidelines 124 from a user interface 250 or other means. The safety guidelines may be stored in the memory 175 of the controller 120. The safety guidelines 124 may contain data regarding the amount of disinfecting light 102 may be safely emitted over a period of time. The controller 120 may compensate for these safety guidelines 124 by adjusting the generated panel control signal 122 to partially dim (such as a dimming level 128 of 50 percent) a switchable mirror panel 112 such that the disinfecting light 102 emitted by the panel conforms with the safety guidelines 124.

Further, the controller 120 may receive information regarding the intensity 126 of the disinfecting light 102 from the user interface 250 or other means, such as a detector circuit electrically coupled to the disinfecting light 102 and configured to automatically determine the intensity 126 of the disinfecting light 102. The intensity 126 may be stored in the memory 175 of the controller 120. If the intensity 126 of the disinfecting light 102 is higher than desired, the controller 120 may compensate adjusting the generated panel control signal 122 to partially dim (such as a dimming level of 25 percent) one or more switchable mirror panels 112.

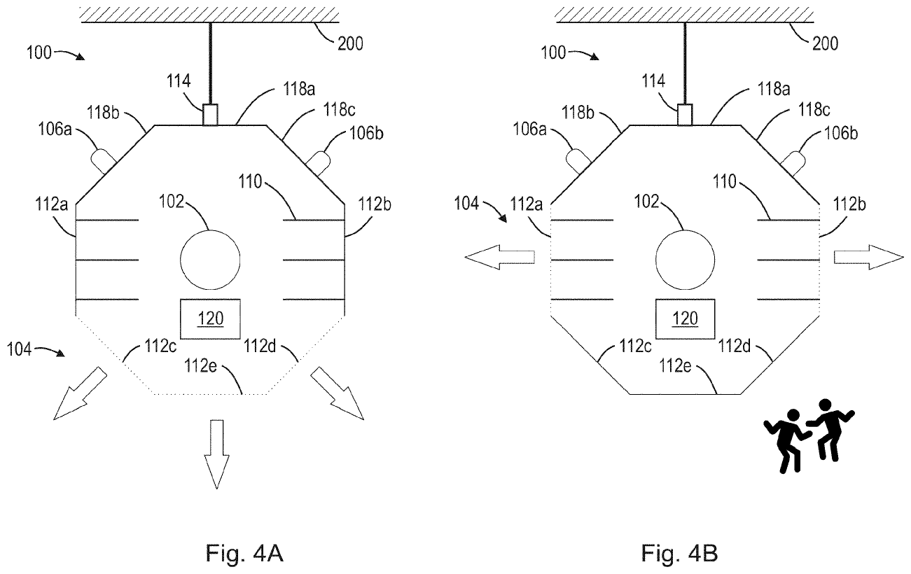

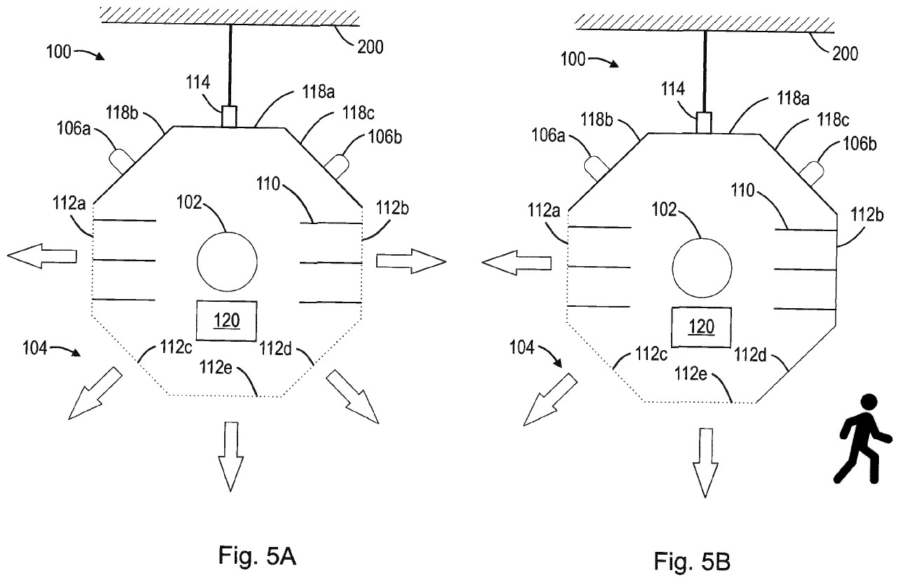

FIGS. 4A, 4B, 5A, and 5B illustrate disinfecting light apparatus 100 switching between modes of operation. In FIG. 4A, disinfecting light apparatus 100 is suspended from ceiling 200 via pendant mount 114. In this example, the occupancy sensors 106a, 106b do not detect any persons in their respective fields of view. Accordingly, the disinfecting light apparatus 100 operates in a “Surface Disinfection” mode. In Surface Disinfection mode, the switchable mirror panels 122c, 122d, 122e of the octagonal disinfecting light apparatus 100 are set to a dimming level of 0 percent. Accordingly, in Surface Disinfection mode, all disinfecting light 104 generated by the disinfecting light source 102 is directed downwards to surfaces below the disinfecting light apparatus 100. These surfaces could include furniture, fixtures, floors, or any other surface which may require disinfecting.

In FIG. 4B, at least one of the occupancy sensors 106a, 106b detects the individuals below the disinfecting light apparatus 100. The occupancy signals 108a, 108b are provided to controller 120, which switches the disinfecting light apparatus 100 to “Upper-Air Disinfection” mode. In response to receiving the occupancy signals 106a, 106b, the controller 120 generates panel control signals 122c, 122d, 122e to set the dimming levels 128c, 128d, 128e of the lower switchable mirror panels 112c, 112d, 112e to 100 percent. Simultaneously, the controller 120 also generates panel control signals 122a, 122b to set the dimming levels 128a, 128b of the side switchable mirror panels 112a, 112b to 0 percent. Accordingly, in Upper-Air Disinfection mode, all disinfecting light 104 is directed horizontally relative to the disinfecting light apparatus 100, thus disinfecting the upper air of the room in which the apparatus 100 is installed. Using the Upper-Air Disinfection mode prevents persons from being exposed to potentially harmful disinfecting light 100 without powering the disinfecting light source 102 off.

In FIG. 5A, the lighting apparatus 100 operates in a combined Surface-Upper Air Disinfection mode. In this mode, all of the switchable mirror panels 112a-112e are set to dimming levels 128a-128e of 0 percent. Accordingly, in this mode, the disinfecting light 102 is directed towards both the surfaces below the disinfecting light apparatus 100, as well as the upper air of the room in which the apparatus 100 was installed.

In FIG. 5B, the controller 120 receives occupancy signals 108a, 108b from occupancy sensors 106a, 106b indicative of a person located to below-and-to-the-right of the lighting apparatus 100. Based on this indication, the controller 120 switches the disinfecting light apparatus into “Directional Disinfecting” mode by identifying switchable mirror panels 112b, 112d as controlling the emission of the disinfecting light 104 which would be incident upon the location of the person. In order to prevent the person from receiving dangerous disinfecting light 102, the controller 120 generates panel control signals 122b, 122d to set the dimming levels 128b, 128d of the switchable mirror panels 112b, 122d to 100 percent. In this way, the Directional Disinfecting mode protects the detected person from the disinfecting light 104, while other, non-occupied portions of the environment are disinfected.

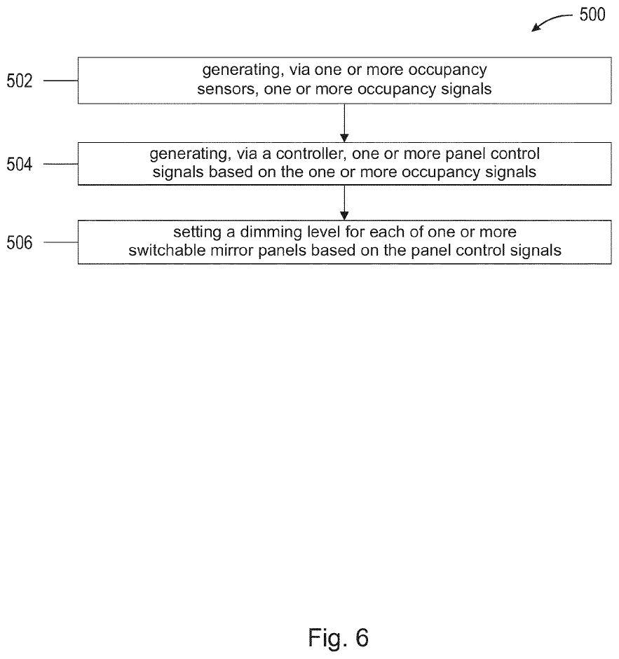

FIG. 6 illustrates a method 500 for selectively dimming individual switchable mirror panels of a disinfecting light apparatus. The method 500 includes generating 502, via one or more occupancy sensors, one or more occupancy signals. The occupancy signals may be based on a location of one or more persons within fields of view of one or more of the occupancy sensors. The method 500 further includes generating 504, via a controller, one or more panel control signals based on the one or more occupancy signals. The method 500 further includes setting 506 a dimming level for each of one or more switchable mirror panels based on the panel control signals. This method 500 enables the disinfecting light apparatus to act in Directional Disinfection mode by controlling the dimming levels of the switchable mirror panels based on the location of persons in the fields of view of the occupancy sensors.

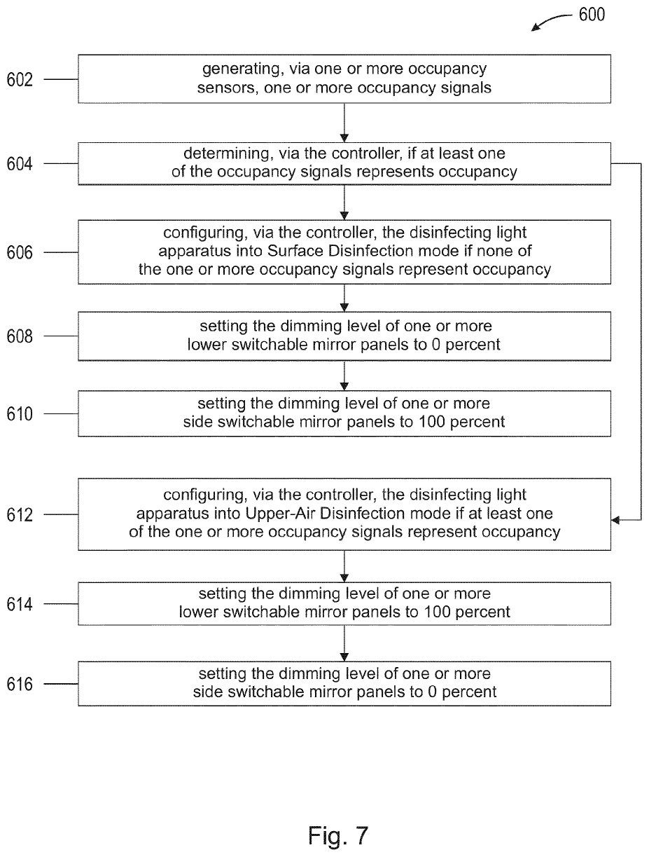

FIG. 7 illustrates a method 600 for transitioning a disinfecting light apparatus between Surface Disinfection mode and Upper-Air Disinfection mode. The method 600 includes generating 602, via one or more occupancy sensors, one or more occupancy signals. The method further includes determining 604, via the controller, if at least one of the occupancy signals represents occupancy.

The method 600 further includes configuring 606, via the controller, the disinfecting light apparatus into Surface Disinfection mode if none of the one or more occupancy signals represent occupancy. In Surface Disinfection mode, the method 600 further includes setting 608 the dimming level of one or more lower switchable mirror panels to 0 percent, and setting 610 the dimming level of one or more side switchable mirror panels to 100 percent.

The method 600 further includes configuring 612, via the controller, the disinfecting light apparatus into Upper-Air Disinfection mode if at least one of the one or more occupancy signals represent occupancy. In Upper-Air Disinfection mode, the method 600 further includes setting 614 the dimming level of one or more lower switchable mirror panels to 100 percent, and setting 616 the dimming level of one or more side switchable mirror panels to 0 percent.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure

1. A disinfecting light apparatus, comprising:
a disinfecting light source configured to generate disinfecting light;
one or more occupancy sensors, wherein each occupancy sensor is configured to generate an occupancy signal corresponding to an occupancy state within a field of view of the respective occupancy sensor;
one or more reflective louvers configured to direct the disinfecting light horizontally relative to the disinfecting light apparatus; and
one or more switchable mirror panels, wherein each switchable mirror panel is configured to dim disinfecting light from the disinfecting light source according to at least one of the one or more occupancy signals, wherein the occupancy signals are used to determine a dimming level of respective ones of the one or more switchable mirror panels.

2. The disinfecting light apparatus of claim 1, wherein the disinfecting light is germicidal ultraviolet (GUV) light.

3. The disinfecting light apparatus of claim 1, wherein at least one of the one or more occupancy sensors are passive infrared (PIR) sensors.

4. The disinfecting light apparatus of claim 1, further comprising one or more pendant mounts configured to mount the disinfecting light apparatus to a ceiling.

5. The disinfecting light apparatus of claim 1, wherein the disinfecting light apparatus is approximately octagonal.

6. The disinfecting light apparatus of claim 5, wherein the disinfecting light apparatus comprises 5 to 7 switchable mirror panels.

7. The disinfecting light apparatus of claim 1, wherein the one or more reflective louvers are positioned adjacent to at least one of the one or more switchable mirror panels.

8. The disinfecting light apparatus of claim 7, wherein the one or more reflective louvers are arranged at an angle of 89 to 91 degrees relative to an adjacent switchable mirror panel.

9. The disinfecting light apparatus of claim 1, further comprising a metal top cover.

10. The disinfecting light apparatus of claim 9, wherein the metal top cover is adjacent to at least one of the one or more switchable mirror panels.

11. The disinfecting light apparatus of claim 1, further comprising a controller configured to generate a panel control signal based on at least one of the one or more occupancy signals, wherein the panel control signal sets a dimming level for at least one of the switchable mirror panels.

12. The disinfecting light apparatus of claim 11, wherein the panel control signal sets the dimming level of the at least one of the one or more switchable mirror panels between 0 and 100 percent.

13. The disinfecting light apparatus of claim 12, wherein at least one of the one or more switchable mirror panels dim to 100 percent if at least one of the one or more occupancy signals indicates occupancy.

14. The disinfecting light apparatus of claim 11, wherein the panel control signal is further based on one or more safety guidelines indicating an amount of disinfecting light to be emitted by the light sources.

15. The disinfecting light apparatus of claim 11, wherein the panel control signal is further based on the intensity of the disinfecting light.
